(19) United States
(12) Patent Application Publication
(10) Pub. No.: US 2007/0270854 A1
(43) Pub. Date: Nov. 22, 2007

(54) Bone anchoring device

(76) Inventors: Zhigang Li, Hillsborough, NJ (US); Kevin Cooper, Flemington, NJ (US); Raymond S. Shissias, Iselin, NJ (US); Richard Pellegrino, Upton, MA (US); Douglas R. LaSota, Saugus, MA (US); Jonathan Fanger, Fall River, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/393,504

(22) Filed: Mar. 30, 2006

(51) Int. Cl. A61B 17/58

(52) U.S. Cl. 606/72

(57) ABSTRACT

A bone anchoring device for securing suture or cable within a bone hole of a bone includes an anchor member and a cable. The anchor member includes engagement ribs and an axial cutout. The axial cutout further includes a cutout surface and at least one pin that extends radially outward from the cutout surface. The cable is secured to the anchor member by penetrating the cable with the pins that extend radially from the axial cutout surface, such that the cable distal end aligns with the anchor distal end and that the cable proximal end extends beyond the anchor proximal end. This results in a bone anchoring device in which failure due to low pullout strength of the cable is greatly minimized or eliminated.

### FIELD OF THE INVENTION

The present invention relates to bone anchoring devices and their methods of use. More specifically, the invention relates to an anchor for securing a cable within a hole or opening in a bone, and, a method of attaching the cable to the anchor.

### BACKGROUND OF THE INVENTION

A wide variety of techniques are available to surgeons for securing sutures or cables within a hole or opening in a bone. Conventionally, such an opening may be a bone tunnel with open ends or a bore hole with one open end and an opposed closed end. Screws, rivets, vertebral straps, suture anchors, and other types of interference fitting anchors are commonly used. In all cases a solid and secure attachment between the suture, or cable, and the anchoring member for anchor devices is key.

The prior art is replete with implantable bone fasteners that include screw members having a threaded body. Typically, the cable, or suture, may be directly attached to the screw member in a variety of conventional manners including threading the suture through an internal channel in the body of the screw or threading the suture through an attached eyelet. One type of known bone fastener includes an expandable member having an axial channel and an elongated insertion element insertable therein. When the insertion element is driven into the axial channel in the expandable member, an interference or interlocking fit secures the insertion element to the expander, thereby securing the suture within the bone bore hole. Load forces exerted on the suture act directly on the insertion element so that the security of the suture within the bone bore hole depends on the security of the engagement between the insertion element and the expandable member.

In one known device, an expandable sheath in which the cable load (bearing force) acts directly on an expandable sheath that is expanded by an expander member for an interference fit within a bore hole in a bone. In such a device the cable load (bearing force) acts directly on the expandable sheath so that the fastening strength of the anchor is independent of the axial security of engagement between the expander member and the expandable sheath. Such a bone anchoring device avoids the failure mode of expander separation from the expandable sheath under suture loading.

In general, methods to attach or secure cables to anchoring members are insert molding, or passing the cables through eyelets or small holes in the anchoring members. The disadvantage of the former methods is low pullout strength of the cable from the anchoring member because insert-molding processes cannot form a secure attachment between the cable and anchoring device. In the latter method, the hole or eyelet is related to the removal of material from the anchoring member that may result in mechanical strength lost, or it may be difficult or not possible to place a hole or eyelet at the anchoring member due to a low profile configuration or limited space. Thus, there is a need for a device where the cable can be securely attached without compromising the structural integrity of the anchoring member.

### SUMMARY OF THE INVENTION

The problems and disadvantages of the prior art devices described above are overcome by the present invention through the provision of a novel bone anchoring device. The bone anchoring device has an anchor member, a cable, and a cover. The anchor member is provided with a series of engagement ribs to form an interference fit with the surface of a bone hole and pins for attaching the cable. The cable is attached to the anchor member by penetrating the cable using the pins and securing it in place by welding. A cover may be mounted to the anchoring member, and optionally the cover and pins are welded together.

These and other aspects and advantages of the present invention will become more apparent by the following description and accompanying drawings.

### BRIEF DESCRIPTION OF THE DRAWINGS

FIG. 1A is an exploded perspective view of a bone anchoring device according to one embodiment of the present invention;

FIG. 1B is a perspective view of the bone anchoring device of FIG. 1A, wherein the device is partially assembled;

FIG. 1C is a perspective view of the bone anchoring device of FIG. 1A, wherein the device is fully assembled;

FIG. 2A is a perspective view of an alternate embodiment of an anchoring member;

FIG. 2B is a perspective view of a partially assembled bone anchoring device using the anchoring member of FIG. 2B;

FIG. 2C is a perspective view of the fully assembled bone anchoring device of FIGS. 2A and 2B having an expander in position for expansion;

FIG. 3A is a cross-sectional view of the anchoring device of FIG. 2C prior to deployment in a bone bore hole opening;

FIG. 3B is a cross-sectional view of the device of FIG. 2C, the device being shown in a deployed state within the bone bore hole;

FIG. 4 is a perspective view of a deployment stabilizer unit;

FIG. 5A is a perspective view of a bone-anchoring device assembled with the deployment stabilizer unit of FIG. 4 with an expander in position; and

FIG. 5B is a perspective view of a bone anchoring device fully assembled with the deployment stabilizer unit of FIG. 4 with an expander in position.

### DETAILED DESCRIPTION OF THE INVENTION

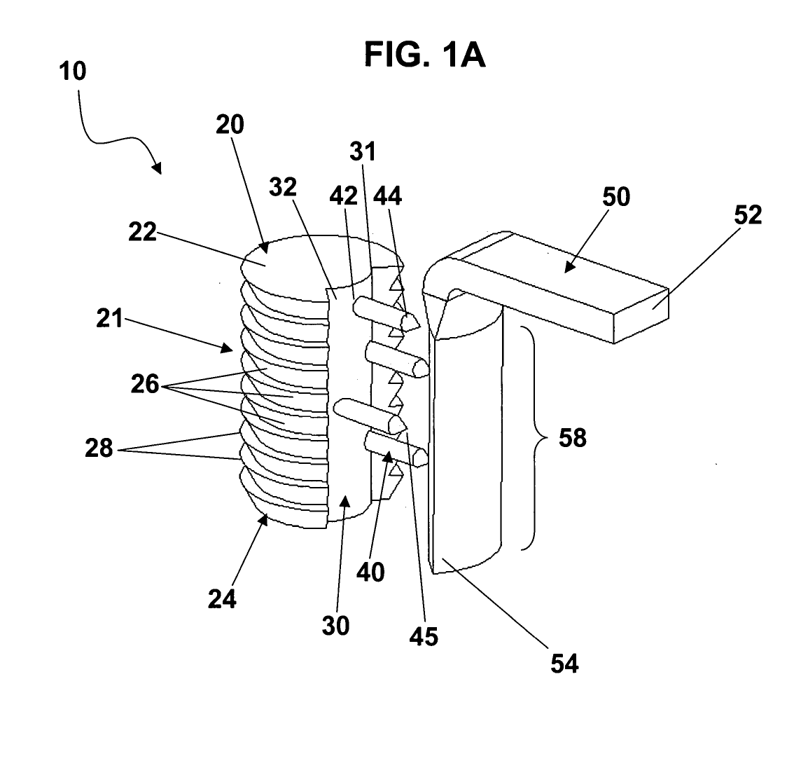

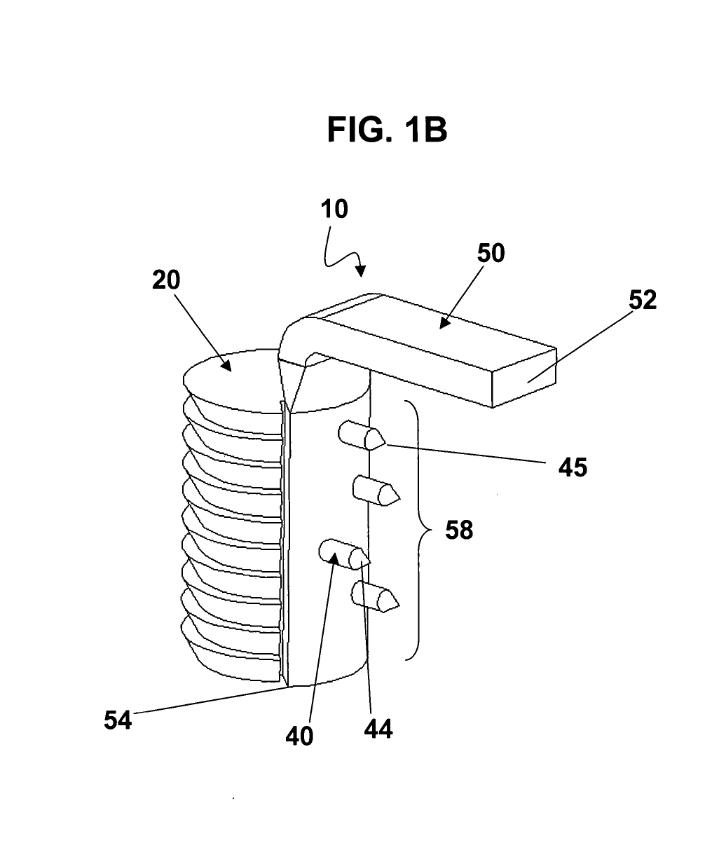

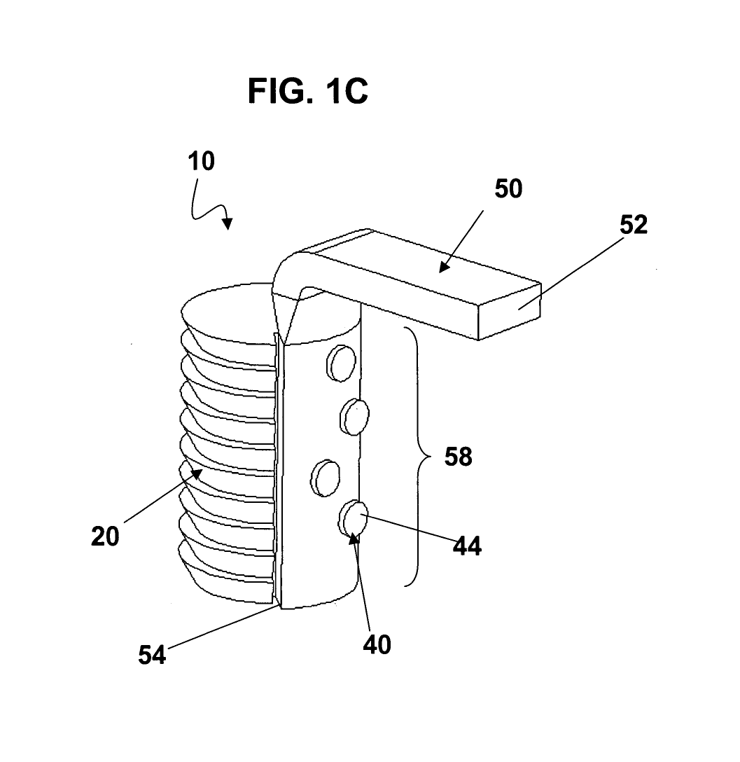

Referring to FIGS. 1A, 1B and 1C there is shown a bone anchoring device 10 for use in surgical procedures in the securing of a cable member 50 to a bone of a patient. The term cable member as used herein refers to a long, generally flat structure such as a solid or perforated polymer ribbon, or a textile structure such as a braided or woven ribbon.

The bone anchoring device 10 includes an anchor member 20 and a cable member 50. Anchor member 20 is seen to have proximal end 22, distal end 24, engagement ribs 26 extending outward from anchor member surface 21 and axial cutout 30 extending into surface 21. Engagement ribs 26 further include bone engagement edges 28. The axial cutout 30 further includes bottom 31 adjacent to cutout surface 32 and at least one pin 40 that extends radially outward from the cutout surface 32. Preferably, and as shown in FIG. 1, a plurality of pins 40 extend radially outward from the cutout surface 32.

Though pins 40 preferably extend perpendicularly from cutout surface 32, one skilled in the art, will appreciate that pins 40 may extend at any angle from cutout surface 32. Though pins 40 are shown in FIG. 1A as randomly located on cutout surface 32, pins 40 may be aligned on cutout surface 32 in a particular pattern or configuration.

Each pin 40 is seen to have a proximal end 42 and a distal end 44. The distal ends are seen to have optional pointed piercing tips 45, but the tips may have any configuration including blunt, rounded, squared-off, and the like. Cable 50 is seen to have proximal end 52 and distal end 54. In the embodiment shown in FIG. 1 cable 50 is tapered, going from thinner at the distal end 54 to thicker at proximal end 52. As shown in FIGS. 1B and 1C, cable member 50 is secured to anchor 20 by penetrating cable 50 with pins 40, i.e., moving the pins 40 through cable 50, such that a section 58 of cable 50 is substantially contained within cutout 30 and substantially in contact with surface 32 as shown in FIG. 1B. Prior to penetration, cable 50 is oriented with anchor 20 such that distal end 54 of cable 50 aligns with distal end 24 of anchor 20, and such that proximal end 52 of cable 50 extends beyond proximal end 22 of anchor 20. Cable 50 and anchor 20 are secured in a variety of conventional manners, preferably by welding pins 40 to cable 50, resulting in the deformation of the distal ends 44 of pins 40 as shown in FIG. 1C. The bone anchoring devices 10 of the present invention eliminates or greatly minimizes any incidence of anchor failure due to low pullout strength of the cable from the anchor. Additionally, in another embodiment of the current invention (not shown), the anchor 20 and cable 50 are further secured with a cover mounted flush over cable 50. This offers the further advantage that cable 50 is protected from damage by bone during the insertion process by the cover.

Cable member 50 may be tipped as described below to reinforce the section of cable member 50 attached to anchor member 20 of bone anchoring device 10. The purpose of tipping cable member 50 is to encapsulate the end of the cable with a material, such as a polymer, for a predetermined length, typically as long as the section to be attached to the anchor member 20. During the tipping operation, which can be done by several methods including compression and injection molding, holes for pins 40 on anchor member 20 are incorporated. This is done by having posts in the tipping mold to create the holes in cable member 50. During the tipping process these holes are permanently formed in cable member 50. The tipping process may strengthen the cable member 50 to help eliminate “cheese wire” damage to cable member 50 under axial loads.

The bone anchoring devices 10 of the present invention may be used to secure suture or cable within a bone bore hole for a variety of uses in surgical procedures. Typically, the surgeon will form a pilot hole for device 10 using a conventional surgical drill. The diameter of the pilot hole is preferably slightly smaller than the diameter of anchoring device 10. Device 10 would then be deployed in the pilot hole through the application of force resulting in the device being maintained in the pilot hole via an interference fit.

The anchoring devices of the present invention have a variety of uses in surgical procedures. These uses include reattachment of ligaments or tendons to bone. Optionally, cable member 50 of bone anchoring device 10 could be connected to a second bone anchor (not shown), which would be secured within a second bore hole in bone. This arrangement could be used, for example, to hold a bone block between adjacent vertebrae in spinal fusion procedures.

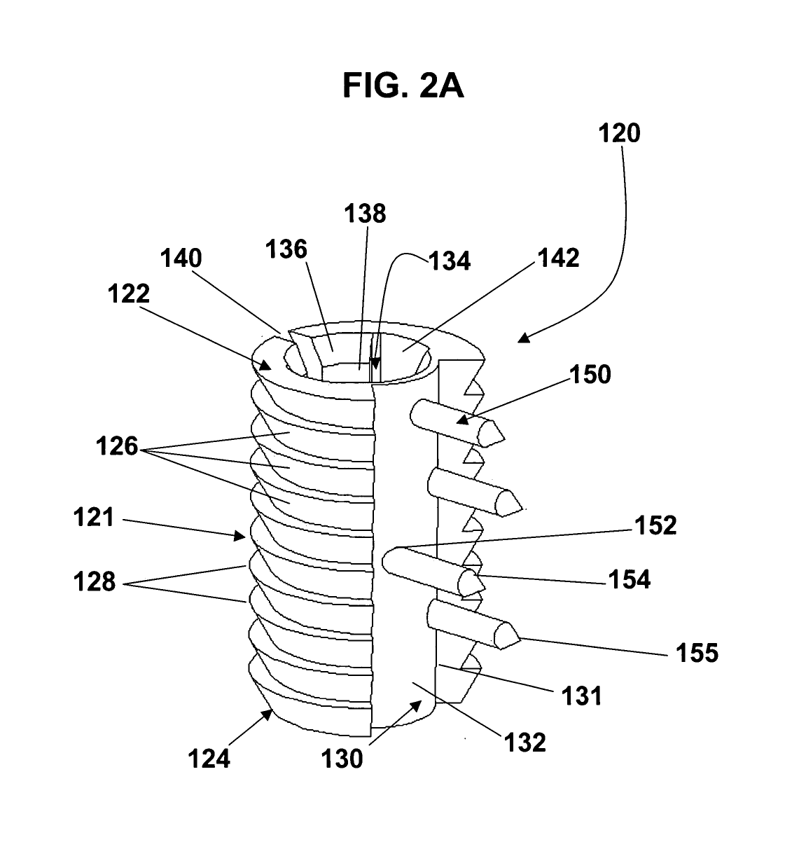

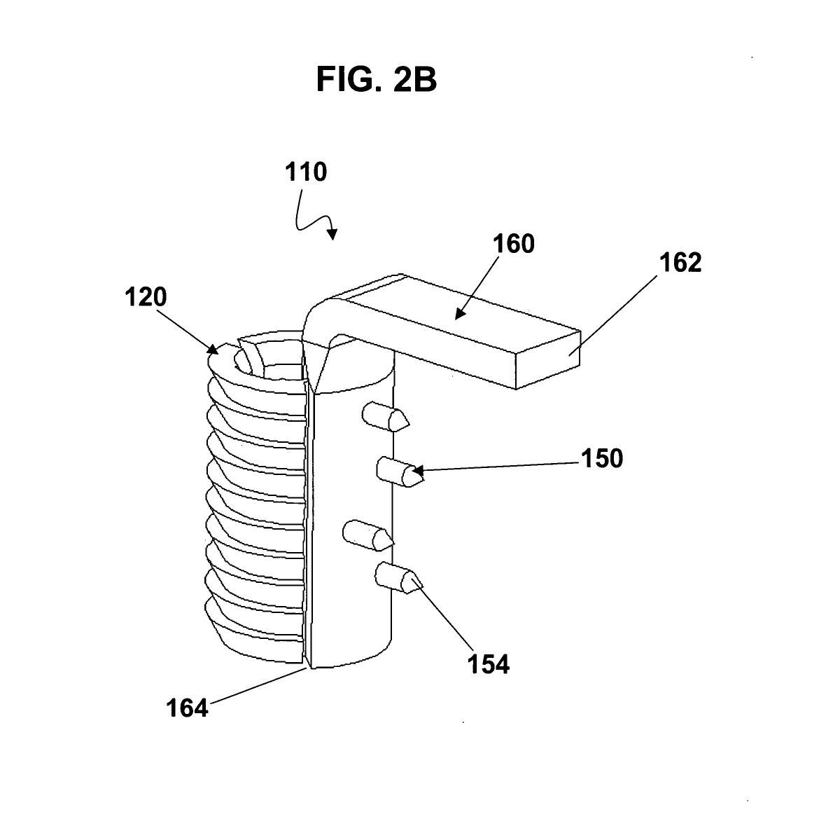

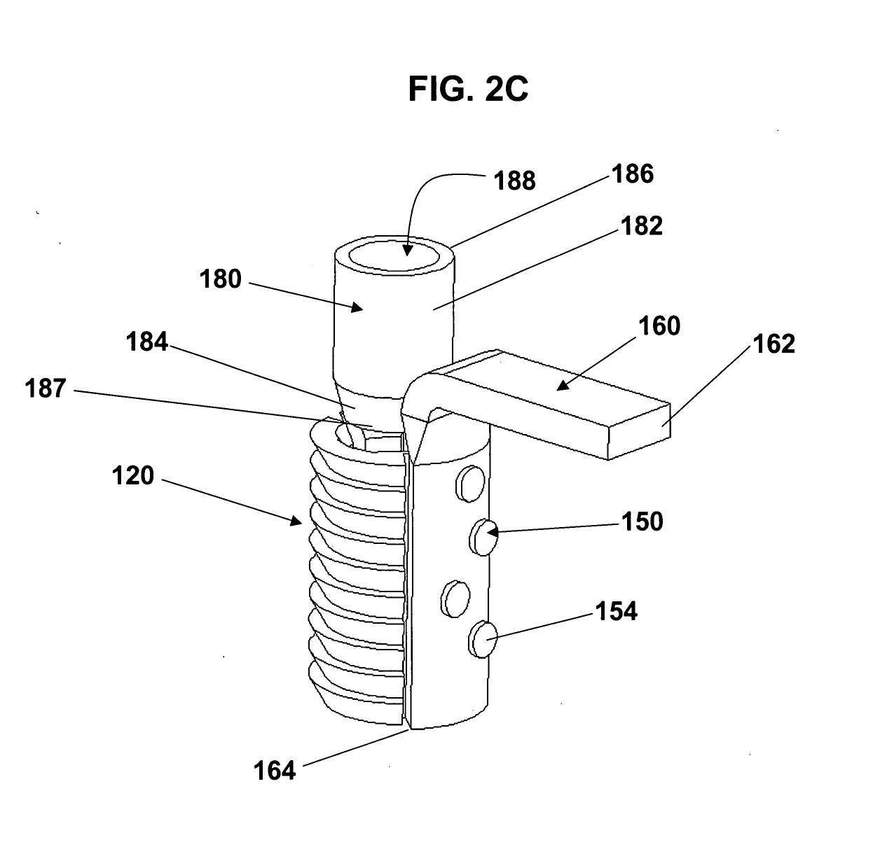

Referring to FIGS. 2A, 2B and 2C, an alternative embodiment of a device of the present invention is shown having an expandable anchor member 120. Expandable anchor member 120 is seen to have proximal end 122, distal end 124, engagement ribs 126 extending out from surface 121, axial cutout 130, axial passageway 134, chamfered or beveled edge 136, inner wall surface 138 and axial slot 140. Axial slot 140 has width W. The engagement ribs 126 have engagement edges 128. The axial cutout 130 extending into surface 121 is seen to have bottom 131 adjacent to surface 132. A plurality of pins 150 is seen extending radially outward from the cutout surface 132, preferably perpendicularly from cutout surface 132. Pins 150 are seen to have proximal ends 152 and distal ends 154. Each pin 150 is seen to have distal piercing tip 155, but the tip 150 may have any configuration including blunt, rounded, squared-off, and the like.

Optionally, expandable anchor member 120 contains a wall hinge to aid in the expansion of expandable anchor member 120. Preferably, the wall hinge is an inner wall hinge 142 (see FIG. 2A).

As shown in FIGS. 2B and 2C, cable member 160 is secured to expandable anchor member 120 by penetrating cable 160 with pins 150 i.e., moving the pins 150 through the cable 160, such that cable 160 is substantially contained in cutout 130 and substantially in contact with cutout surface 132. Prior to penetration, cable 160 is oriented with expandable anchor 120 such that distal end 164 of cable 160 aligns with distal end 124 of anchor member 120, and such that proximal end 162 of cable 160 extends beyond proximal end 122 of anchor member 120. Cable 160 and expandable anchor member 120 may be secured together in a variety of conventional ways, including welding pins 150 to cable 160, resulting in the deformation of distal ends 154 of pins 150 as seen in FIG. 2C. This combination of elements results in an assembled bone anchoring device 110 in which anchor failure due to low pullout strength of the cable is greatly minimized or prevented. In an additional embodiment of the current invention (not shown), expandable anchor member 120 and cable 160 are further secured with a cover mounted flush over cable 160. This offers the further advantage that cable 160 is protected from damage by bone during the insertion process by the cover.

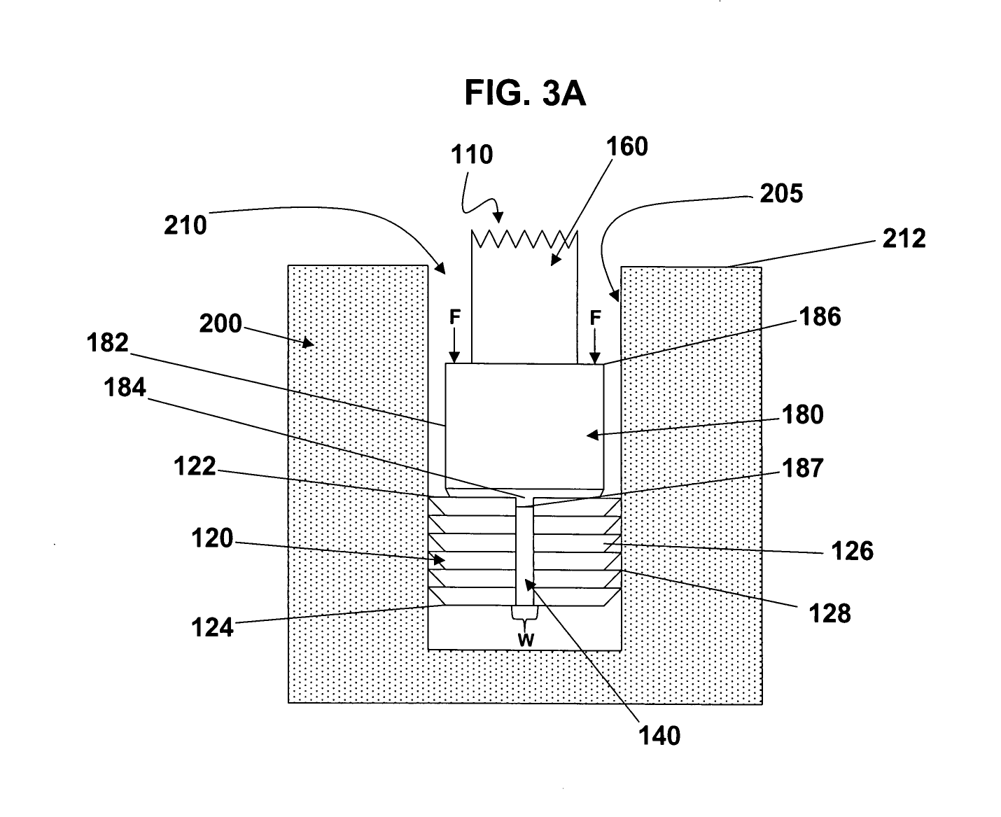

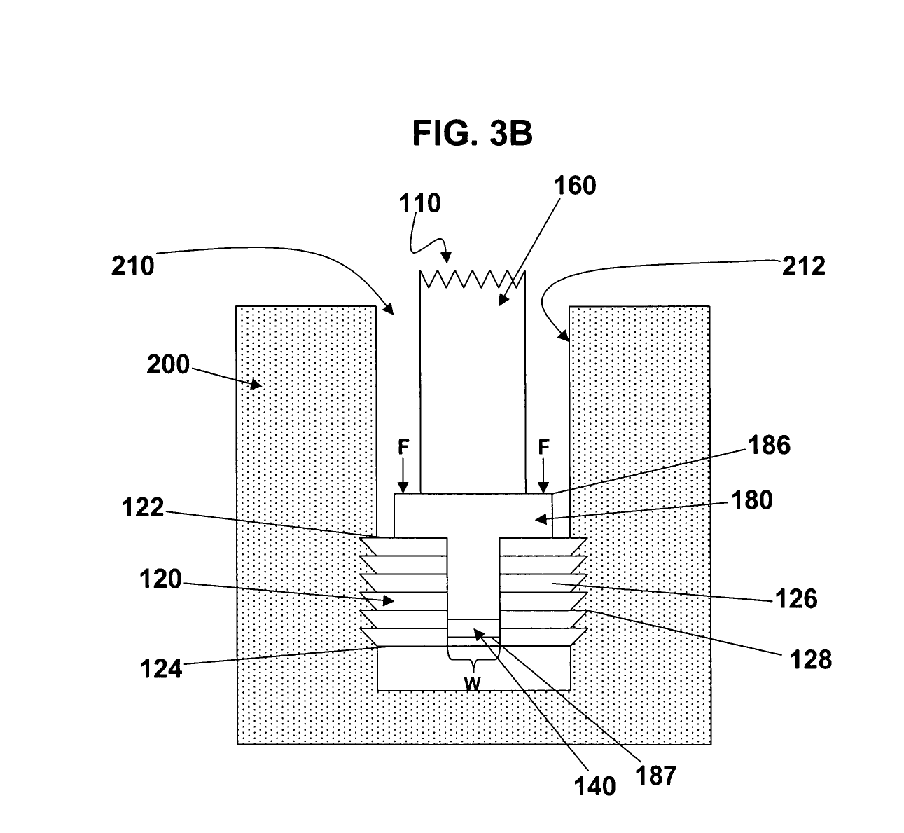

FIG. 2C shows a fully assembled bone-anchoring device 110 according to one embodiment of the present invention with expander member 180 in deployment position. Expander member 180 is generally cylindrically-shaped and includes an outer wall surface 182, a proximal end 186 and a distal end 187. The expander member 180 also includes a chamfered or beveled edge 184 located at the distal end 187 of the expander member 180. The chamfered edge 184 is used to serve as a lead-in for the initial insertion of the expander member 180 into the expandable anchor 120 as shown in FIG. 3A.

FIGS. 3A and 3B demonstrate the expansion of the bone anchoring device 110 for an interference fit in a bone bore hole. This expansion of the bone anchoring device 110 is achieved by the inward driving of the expander member 180 by a force F into the axial passageway 134 of the expandable anchor member 120. The interference fit between the outer wall surface 182 of expander member 180 and the inner wall surface 138 of the axial passageway 134 of anchor member 120 forces the anchor 120 to expand radially to conform the inner diameter of passageway 134 to the outer diameter of expander member 180. The expandable anchor member 120 expands radially due to the further separation of slot 140 with the full insertion of the expander member 180 within the axial passageway 134 of the expandable anchor member 120. When the bone anchoring device 110 is fully deployed, as shown in FIG. 3B, slot 140 has increased in width W.

FIGS. 3A and 3B show the initial and final configurations, respectively, of the bone anchoring device 110 when deployed in a bone bore hole 210 in order to anchor the cable member 160 to bone 200.

FIG. 3A shows the initial deployment configuration of the bone anchoring device 110, which demonstrates the placement of the expander member 180 and the expandable anchor 120 within the bone bore hole 210. The diameter of the bone bore hole 210 is equal to or only slightly larger than the outer diameter of bone anchoring device 110. The bone anchoring device 110 is placed within the bone hole 210 such that the proximal surface end 186 of the expander member 180 is flush or below surface 212 of bone 200 (see FIG. 3A). The chamfered edge 184 of the expander member 180 is in contact with the chamfered edge 136 of the anchor 120 in the initial configuration (expander member 180 has not been deployed within anchor 120). The expander member 180 is then forcibly driven into the axial passageway 134 of the expandable anchor member 120 until full deployment is achieved as shown in FIG. 3B.

With reference to FIG. 3B, the bone anchoring device 110 is shown in its full deployment and final configuration. The expandable anchor 120 is shown expanded to a diameter to interfere with or engage the interior bone surface 205 of the bone bore hole 210 allowing the engagement edges 128 of ribs 126 to engage and cut into bone 200. FIG. 3B also shows the increasing of width W of slot 140 when deployment of the expander member 180 within the expandable anchor 120 is completed. The expansion of slot 140 allows uninhibited circumferential expansion of the expandable anchor 120 for accommodating the circumference of the expander member 180. The advantage of slot 140 is that it provides for the uniform radial expansion along the entire length of expandable anchor 120 and thus, allows for relatively very large radial and elastic expansion of anchor 120.

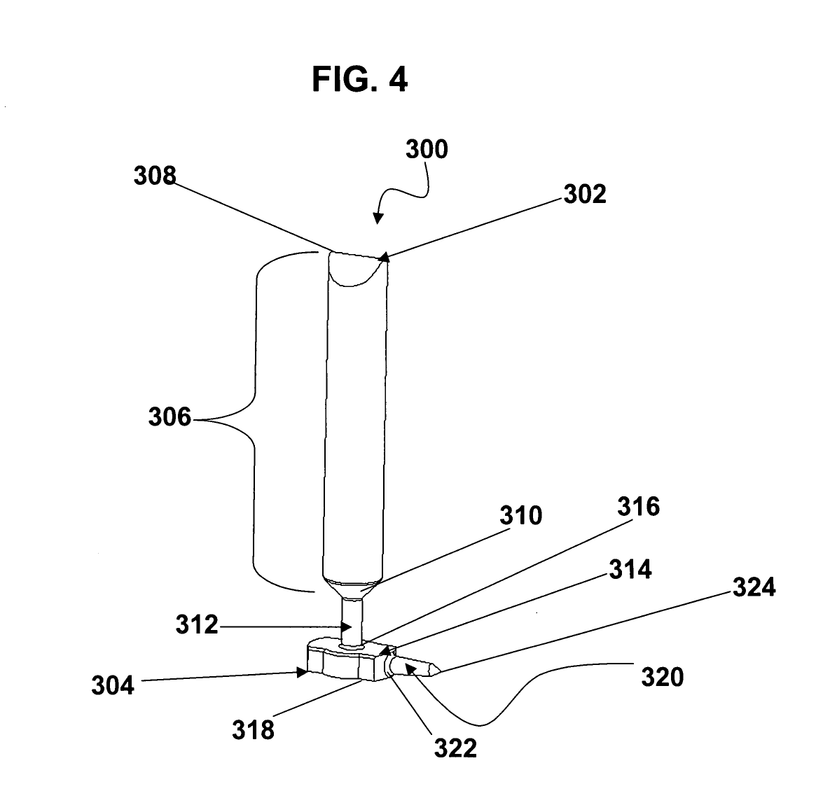

Deployment stabilization unit (DSU) 300 is seen in FIG. 4. DSU 300 contains unit proximal end 302, unit distal end 304, post 306, frangible break-away section 312, and stabilization seat 314. Post 306 further contains post proximal end 308 and post distal end 310. Additionally, stabilization seat 314 contains seat proximal end 316, seat distal end 318 and seat attachment pin 320 with seat pin proximal end 322 and seat pin distal end 324.

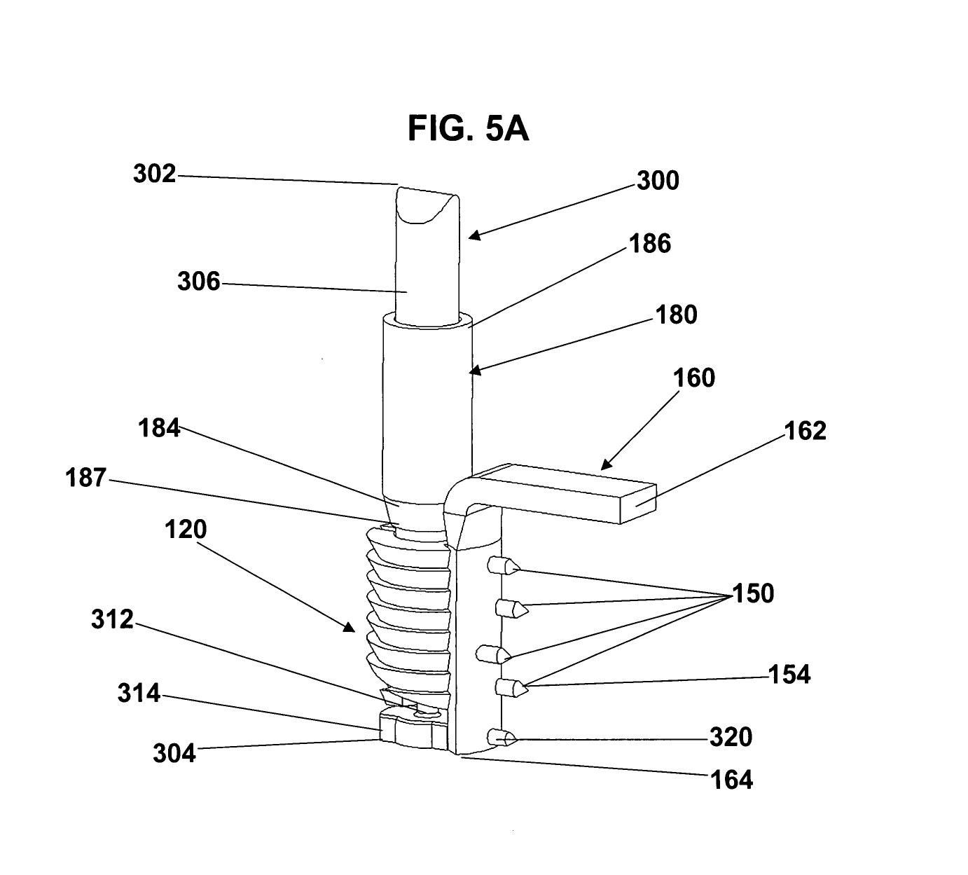

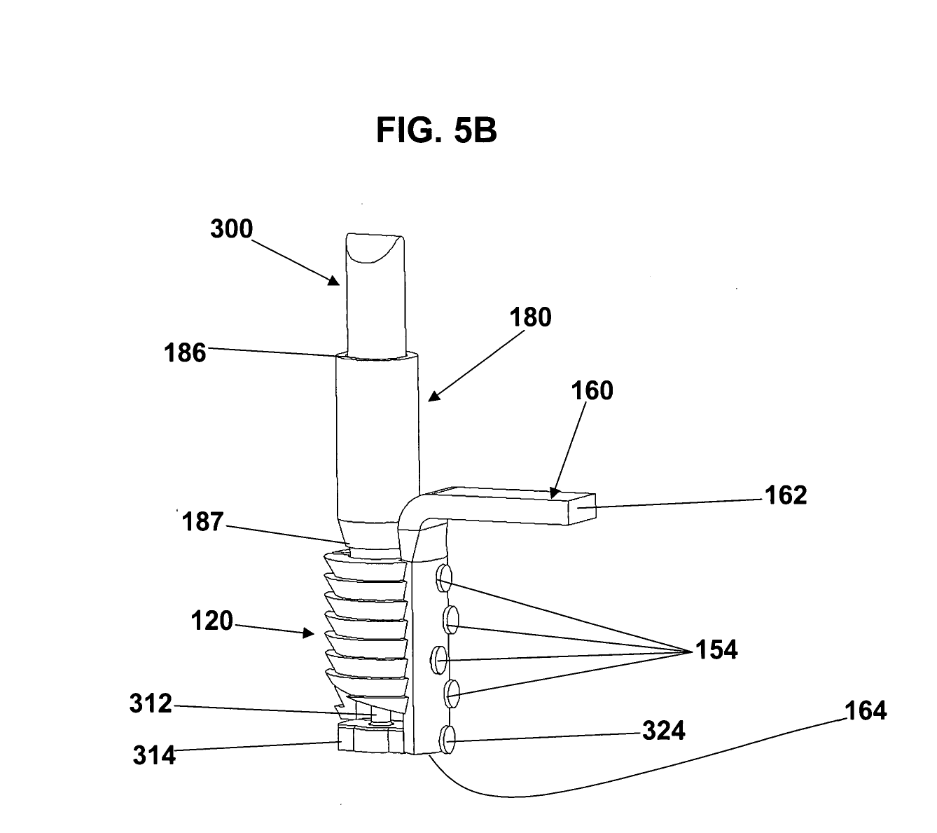

Bone anchoring device 110, is shown in combination with expander 180 and DSU 300 in FIGS. 5A and 5B. DSU 300 is inserted through anchor member 120 by way of axial passageway 134 such that DSU seat proximal end 316 is flush with expandable anchor distal end 124, and that expandable anchor pins 150 are aligned with seat attachment pin 320.

As shown in FIGS. 5A and 5B, cable member 160 is secured to expandable anchor member 120 and DSU 300 by penetrating cable 160 with anchor pins 150 and seat pin 320 from pins distal ends 154 and 324 to pins proximal ends 152 and 322, such that cable 160 is flush with cutout surface 132. Prior to penetration, cable 160 is oriented such that cable distal end 164 aligns with DSU distal end 304, and that cable proximal end 162 extends beyond anchor proximal end 122. Cable 160, expandable anchor 120 and DSU 300 are secured by the welding of anchor pins 150 and seat attachment pin 320 to cable 160, resulting in the deformation of pins distal ends 154 and 324 shown in FIG. 5B. This results in bone anchoring device 110 in which anchor failure due to low pullout strength of the cable is substantially eliminated. Additionally, in an alternate embodiment of the current invention (not shown) where anchor 120 and cable 160 are further secured with a cover mounted flush over cable 160 and anchor 120. This offers the further advantage that cable 160 is protected from damage by bone during the insertion process by the cover.

Use of DSU 300 in combination with bone anchoring device 110 is advantageous because the forces on DSU 300 counter the forces on the expander element (see FIGS. 3A and 3B), thus ensuring that bone anchoring device 110 does not migrate in the bone hole 210. After deployment DSU post 306 of DSU 300 is removed. This is accomplished though the breakage of DSU break-away section 312 either during expander 180 deployment or immediately there after by applying torque to DSU post 306 (not shown).

Uses of bone anchoring device 110 include reattachment of ligaments or tendons to bone. Furthermore, cable member 160 of bone anchoring device 110 could be connected to a second bone anchoring device (not shown), which is secured within a second hole opening in bone. This arrangement could be used, for example, to hold a bone block between adjacent vertebrae in spinal fusion procedures.

Bone anchoring device 110, expander 180 and deployment stabilization unit (DSU) 300 may be available separately, or can be in combination as part of a bone anchoring kit. The pieces may be fully, or partially, assembled prior to use, and preferably packaged and sterilized prior to being made available.

Suitable materials from which bone anchoring devices 10 and 110 may be formed include biocompatible polymers such as aliphatic polyesters, polyorthoesters, polyanhydrides, polycarbonates, polyurethanes, polyamides and polyalkylene oxides. The present invention also can be formed from absorbable glasses or ceramics comprising calcium phosphates and other biocompatible metal oxides (i.e., CaO), metals, combinations of metals, autograft, allograft, or xenograft bone tissues.

In the preferred embodiment, the bone anchoring device is formed from aliphatic polyester polymers, copolymers or blends thereof. The aliphatic polyesters are typically synthesized in a ring opening polymerization. Suitable monomers include but are not limited to lactic acid, lactide (including L-, D-, meso and D,L mixtures), glycolic acid, glycolide, epsilon-caprolactone, p-dioxanone(1,4-dioxan-2-one), trimethylene carbonate(1,3-dioxan-2-one), delta-valerolactone, beta-butyrolactone, epsilon-decalactone, 2,5-diketomorpholine, pivalolactone, alpha-diethylpropiolactone, ethylene carbonate, ethylene oxalate, 3-methyl-1,4-dioxane-2,5-dione, 3,3-diethyl-1,4-dioxan-2,5-dione, gamma-butyrolactone, 1,4-dioxepan-2-one, 1,5-dioxepan-2-one, 6,6-dimethyl-dioxepan-2-one, 6,8-dioxabicycloctane-7-one and combinations thereof. These monomers generally are polymerized in the presence of an organometallic catalyst and an initiator at elevated temperatures. The organometallic catalyst is preferably tin based, e.g., stannous octoate, and is present in the monomer mixture at a molar ratio of monomer to catalyst ranging from about 10,000/1 to about 100,000/1. The initiator is typically an alkanol (including diols and polyols), a glycol, a hydroxyacid, or an amine, and is present in the monomer mixture at a molar ratio of monomer to initiator ranging from about 100/1 to about 5000/1. The polymerization typically is carried out at a temperature range from about 80° C. to about 240° C., preferably from about 100° C. to about 220° C., until the desired molecular weight and viscosity are achieved.

In another embodiment of the present invention, the polymers and blends can be used as a therapeutic agent release matrix. Prior to forming the bone anchoring device, the polymer would be mixed with a therapeutic agent. The variety of different therapeutic agents that can be used in conjunction with the polymers of the present invention is vast. In general, therapeutic agents which may be administered via the pharmaceutical compositions of the invention include, without limitation: anti-infectives such as antibiotics and antiviral agents; chemotherapeutic agents (i.e. anticancer agents); anti-rejection agents; analgesics and analgesic combinations; anti-inflammatory agents; hormones such as steroids; growth factors, including bone morphogenic proteins (i.e. BMP's 1-7), bone morphogenic-like proteins (i.e. GFD-5, GFD-7 and GFD-8), epidermal growth factor (EGF), fibroblast growth factor (i.e. FGF 1-9), platelet derived growth factor (PDGF), insulin like growth factor (IGF-I and IGF-II), transforming growth factors (i.e. TGF-beta I-III), vascular endothelial growth factor (VEGF); and other naturally derived or genetically engineered proteins, polysaccharides, glycoproteins, or lipoproteins.

Matrix materials for the present invention may be formulated by mixing one or more therapeutic agents with the polymer. Alternatively, a therapeutic agent could be coated onto the polymer, preferably with a pharmaceutically acceptable carrier. Any pharmaceutical carrier can be used that does not dissolve the polymer. The therapeutic agent may be present as a liquid, a finely divided solid, or any other appropriate physical form. Typically, but optionally, the matrix will include one or more additives, such as diluents, carriers, excipients, stabilizers or the like.

The amount of therapeutic agent will depend on the particular drug being employed and medical condition being treated. Typically, the amount of drug represents about 0.001 percent to about 70 percent, more typically about 0.001 percent to about 50 percent, most typically about 0.001 percent to about 20 percent by weight of the matrix. The quantity and type of polymer incorporated into the drug delivery matrix will vary depending on the release profile desired and the amount of drug employed.

Upon contact with body fluids, the polymer undergoes gradual degradation (mainly through hydrolysis) with concomitant release of the dispersed drug for a sustained or extended period. This can result in prolonged delivery (over, say 1 to 5,000 hours, preferably 2 to 800 hours) of effective amounts (say, 0.0001 mg/kg/hour to 10 mg/kg/hour) of the drug. This dosage form can be administered as is necessary depending on the subject being treated, the severity of the affliction, the judgment of the prescribing physician, and the like. Following this or similar procedures, those skilled in the art will be able to prepare a variety of formulations.

The following examples are illustrative of the principles and practice of this invention, although not limited thereto.

### EXAMPLE 1

Forming cable/cover/anchor assemblies In this example, a general ultrasonic welding process was used to form strap, anchor, and cover assembly.

The cover was formed by compression molding (Model 2696, Carver, Inc., Wabash, Ind.). The material used to form the cover was 95/5 poly(lactide-co-glycolide) (95/5 PLGA) from PURAC (Gorinchem, The Netherlands), with an Inherent Viscosity (I.V.) of 2.33 dl/gm (measured in chloroform at 25° C. and a concentration of 0.1 gm/dl). The cover was cut to a square piece with the dimensions of 2×4.5 millimeter.

The cable was a three dimensional woven cable made using 95/5 poly(lactide-co-glycolide) (95/5 PLGA) fibers. The fibers are sold under the tradename PANACRYL, (Ethicon, Inc., Somerville, N.J.). The cable was 3D woven with 100 Denier fiber and a thickness of 1 millimeter and width of 2 millimeter at Fiber Concepts, Inc. (Conshohocken, Pa.).

The anchor was made using 95/5 poly(lactide-co-glycolide) by injection molding (Model NN35M14, Niigata Machinery Plant, Tokyo, Japan).

The anchor was placed into a fixture. The cable was cut to 25 millimeter in length and placed on the anchor by penetrating the cable using the pins of the anchor. The cover is placed to cover the cable. The horn of an ultrasonic welding machine (Model SureWeld 70, Sonobond, West Chester, Pa.) was applied to the pins and the cover, and melted the pins and the cover. The welding time was set to 2.35 seconds, the holding time was set to 1.5 seconds, and the amplitude was set to 9.

The pullout strength of the assembly was tested. Pullout tests were performed using an Instron 4501 test frame. The anchor was first loaded on a polyurethane foam block with a pre-drilled hole with diameter of 5.1 mm, which was fixed in place by a clamp that allows movement in the X-Y plane but not the Z (pulling) direction. The cable end was held tightly by the grips and then a tensile testing procedure was performed with a cross-head rate of 0.1 millimeter/second. The pullout strength of the control was 12.5 pounds-force (Ibf).

### EXAMPLE 2

Surgical Procedure A patient is prepared for spinal fusion surgery in a conventional manner. The surgery will fuse one or more levels of the spinal column. The patient is anesthetized in a conventional manner. The tissue repair site is accessed by making an incision through the abdominal cavity and dissecting down to the spinal column. A sterile device of the present invention is prepared for implantation into the patient, the device having anchor members mounted to each end of the cable. The operative site is prepared to receive the anchor members of the repair device by dissecting through the ligamentous structure attached to the vertebral bodies of the spinal column that will be fused. A discectomy procedure is performed to remove the disc of the vertebral level to be fused and a bone graft is inserted into the discs space. A bore hole is drilled into the vertebral body above and below the disc space. The anchor bodies are then inserted into drilled bore holes in the adjoining vertebrae to be fused. The cable of the device is used to prevent migration of the bone graft in order to complete the tissue repair. The incision is approximated in a conventional manner using conventional surgical sutures.

The incision is bandaged in a conventional manner, thereby completing the surgical procedure.

The novel devices and method of the present invention provide the patient and surgeon with multiple advantages. The advantages include increased pull-out strength of the anchor member from the cord.

It should be understood that the embodiments described herein are merely exemplary and that a person skilled in the art may make many variations and modifications without departing from the spirit and scope of the present invention. All such variations and modifications are intended to be included within the scope of the invention as defined in the appended claims.

We claim:

1. A bone anchoring device comprising:
a cable member having a first end and a second end;
an anchor member;
an axial cutout in the anchor member having a surface; and,
at least one attachment pin extending from the cutout surface,
wherein at least a section of the cable member is mounted in the axial cutout adjacent to the surface such that said attachment pin extends through said section.

2. The bone anchoring device of claim 1, wherein the cable member is selected from the group consisting of solid polymer ribbons, perforated polymer ribbons, braided ribbons, and woven ribbons.

3. The bone anchoring device of claim 1, wherein the end of the cable is encapsulated in a material.

4. The bone anchoring device of claim 1, comprising more than one attachment pin.

5. The bone anchoring device of claim 1, wherein said attachment pin extends perpendicularly from the cutout surface.

6. The bone anchoring device of claim 1, wherein the device comprises a polymer selected from the group consisting of aliphatic polyester polymer, aliphatic polyester copolymers, and blends thereof.

7. The bone anchoring device of claim 6, wherein the aliphatic polyesters comprise monomers selected from the group consisting of lactic acid, lactide, glycolic acid, glycolide, epsilon-caprolactone, 1,4-dioxan-2-one, and 1,3-dioxan-2-one.

8. A method of securing a cable within a bore hole in a bone, said method comprising the steps of:
providing a bone anchoring device, comprising:
a cable member having a first end and a second end;
an anchor member, having a first diameter;
an axial cutout in the anchor member having a surface; and,
at least one attachment pin extending from the cutout surface,
wherein at least a section of the cable member is mounted in the axial cutout adjacent to the surface such that said attachment pin extends through said section;
drilling a bore hole in a bone, the bore hole having a second diameter, wherein the second diameter is less than or equal to said first diameter; and,
and deploying the bone anchoring device in the bore hole.

9. A bone anchoring device, comprising
a cable member;
an expander member;
an expandable anchor member;
an axial passageway in the anchor member;
an axial slot in the anchor member,
an axial cutout in the anchor member having a surface; and,
at least one attachment pin extending from the surface,
wherein at least a section of the cable member is mounted in the axial cutout adjacent to the surface such that each attachment pin extends through said section.

10. The bone anchoring device of claim 9, wherein the cable member is selected from the group consisting of solid polymer ribbons, perforated polymer ribbons, braided ribbons, and woven ribbons.

11. The bone anchoring device of claim 9, wherein the end of the cable is encapsulated in a material.

12. The bone anchoring device of claim 9, comprising more than one attachment pin.

13. The bone anchoring device of claim 9, wherein the expander member further comprises a wall hinge.

14. The bone anchoring device of claim 9, wherein the expandable anchor member additionally comprises an axial passageway with a chamfered or beveled edge.

15. The bone anchoring device of claim 9, wherein the expander member additionally comprises a distal end with a chamfered or beveled edge.

16. The bone anchoring device of claim 9, wherein each attachment pin extends perpendicularly from the cutout surface.

17. The bone anchoring device of claim 9, wherein the device comprises a polymer selected from the group consisting of aliphatic polyester polymers, aliphatic polyester copolymers, and blends thereof.

18. The bone anchoring device of claim 17, wherein the aliphatic polyesters comprise monomers selected from the group consisting of lactic acid, lactide, glycolic acid, glycolide, epsilon-caprolactone, 1,4-dioxan-2-one, and 1,3-dioxan-2-one.

19. A method of securing a cable within a hole opening in a bone, comprising the steps of
providing a bone anchoring device, comprising
a cable member;
an expander member;
an expandable anchor member;
an axial passageway in the anchor member;
an axial slot in the anchor member;
an axial cutout in the anchor member having a surface; and,
at least one attachment pin extending from the surface,
wherein at least a section of the cable member is mounted in the axial cutout adjacent to the surface such that each attachment pin extends through said section;
drilling a bore hole in a bone having a second diameter equal to or less than the first diameter of the device;
deploying the bone anchoring device in the bore hole; and, forcibly driving the expansion member into the axial passageway of the expandable anchor member.

20. A deployment stabilization unit for a bone anchoring device, comprising
a post having a proximal end and a distal end;
a frangible break-away section;
a stabilization seat; and,
a seat attachment pin extending out from the stabilization seat,
wherein the break-away section connects an end of the post to the stabilization seat such that the post and the stabilization seat may be effectively separated upon the application of a sufficient force.
